(19) United States
(12) Patent Application Publication
(10) Pub. No.: US 2008/0038842 A1
(43) Pub. Date: Feb. 14, 2008

(54) SLITRK1 mutations associated with disorders in the OCD spectrum

(75) Inventors: Allison E. Ashley-Koch, Cary, NC (US); Stephan Zuchner, Durham, NC (US); Michael L. Cuccaro, Durham, NC (US); Margaret A. Pericak-Vance, Chapel Hill, NC (US); Harry Hercules Wrights, Columbia, SC (US)

Correspondence Address: MYERS BIGEL SIBLEY & SAJOVEC, PO BOX 37428, RALEIGH, NC, 27627, US

(73) Assignees: Duke University; The University of South Carolina

(21) Appl. No.: 11/825,489

(22) Filed: Jul. 6, 2007

Related U.S. Application Data
(60) Provisional application No. 60/819,095, filed on Jul. 7, 2006.

(51) Int. Cl. G01N 33/53; G01N 33/00; G01N 33/68

(52) U.S. Cl. 436/501; 436/89; 436/94

(57) ABSTRACT

The present invention provides compositions and methods directed to the use of SLITRK1 (slit and trk like 1) mutations to identify, diagnose, and treat disorders in the obsessive-compulsive disorder spectrum. Specifically, the present invention provides methods of identifying a subject having an increased risk of developing a disorder in the OCD spectrum and/or diagnosing a disorder in the OCD spectrum in a subject by detecting in the subject a mutation in the SLITRK1 gene.

### RELATED APPLICATIONS

This application claims the benefit under 35 U.S.C. §119 of U.S. Provisional Application No. 60/819,095 filed Jul. 7, 2006, the entire disclosure of which is incorporated by reference herein in its entirety.

### STATEMENT OF FEDERAL SUPPORT

The present invention was made, in part, with the support of grant numbers NS049067 and NS036768 from the National Institutes of Health. The United States government has certain rights to this invention.

### FIELD OF THE INVENTION

The present invention provides compositions and methods directed to the use of SLITRK1 mutations to identify, diagnose, and treat disorders in the obsessive-compulsive disorder spectrum.

### BACKGROUND OF THE INVENTION

Trichotillomania (TTM) is a chronic behavior disorder that is characterized by the irresistible, repeated urge to pull out one's hair, and results in noticeable hair loss. Clinically, TTM is classified as an impulse-control disorder and believed to belong to the obsessive compulsive disorder (OCD) spectrum. Because of the physical disfigurement associated with TTM, the emotional toll on patients and their families is substantial. This emotional toll is further burdened because hair pulling is often viewed as a controllable, voluntary act. An additional psychological effect can be low self-esteem, often associated with being shunned by peers and the fear of socializing due to appearance and negative attention they may receive. The average age of onset of TTM is 11 to 13 years, with a slightly higher prevalence observed in females as compared with males. Prevalence estimates of TTM range from 0.6% to 3.4%, depending on whether strict or broad clinical criteria are considered.

The SLITRK1 (Slit and Trk-like family member 1) gene encodes a single-pass transmembrane protein with two leucine rich repeat (LRR) motifs in the extracellular domain. SLITRK1 has high relative expression in brain regions implicated with Tourette's Syndrome (TS) and has been suggested to have a role in neurite outgrowth. Rare sequence variants in SLITRK1 are associated with TS, which belongs to the OCD spectrum (J F Abelson, et al. (2005) Science. 310(5746):317-20). We investigated whether sequence variation in the SLITRK1 gene may also contribute to TTM and discovered a correlation between two mutations in the SLITRK1 gene and disorders in the OCD spectrum (S Zuchner, et al. (2006) Molecular Psychiatry. 11:888-889, the entire disclosure of which is incorporated by reference herein in its entirety). These mutations were not identified in over 2,000 control subjects.

There is a need in the art for means of identifying and diagnosing patients having an increased risk of developing a disorder in the OCD spectrum. The present invention addresses this need by providing methods and compositions for identifying, diagnosing, and treating disorders in the obsessive-compulsive disorder spectrum.

### SUMMARY OF THE INVENTION

In one aspect, the invention encompasses a method of identifying a subject having an increased risk of developing a disorder in the OCD spectrum by detecting in the subject a mutation in the SLITRK1 gene between the coding sequence for the second leucine rich repeat (LRR) domain and the transmembrane domain of the SLITRK1 protein.

In another aspect, the invention encompasses a method of diagnosing a disorder in the OCD spectrum in a subject, the method comprising detecting in the subject a mutation in the SLITRK1 gene between the coding sequence for the second leucine rich repeat (LRR) domain and the transmembrane domain of the SLITRK1 protein. In another aspect, the invention further encompasses such a method wherein the mutation is a missense mutation. Such missense mutation may include a change at position 584 and/or position 593 of the SLITRK1 amino acid sequence of SEQ ID NO:1 or a corresponding position in other SLITRK1 proteins. Such mutation may further include a R→K mutation at position 584 and/or an S→G mutation at position 593 of the SLITRK1 amino acid sequence provided in SEQ ID NO:1 or a corresponding position in other SLITRK1 proteins. Such mutation may further include the mutation comprises a G→A mutation at nucleotide 1751 and/or an A→G mutation at nucleotide 1777 of the SLITRK1 nucleotide sequence provided in SEQ ID NO:2 or a corresponding position in other SLITRK1 genes.

In another aspect, the invention encompasses a method of identifying a subject having an increased risk of developing a disorder in the OCD spectrum by correlating the presence of one or more mutations in the SLITRK1 gene with an increased risk of developing the disorder and detecting the one or more mutations in the subject, thereby identifying the subject as having an increased risk of developing the disorder. In particular embodiments, the disorder in the OCD spectrum is not Tourette's syndrome.

In another aspect, the invention encompasses a method of diagnosing a disorder in the OCD spectrum in a subject by correlating the presence of one or more mutations in the SLITRK1 gene with an increased risk of developing the disorder and detecting the one or more mutations in the subject, thereby diagnosing the subject as having the disorder. In particular embodiments, the disorder in the OCD spectrum is not Tourette's syndrome.

In another aspect, the invention encompasses a method of identifying a subject having an increased risk of developing a disorder in the OCD spectrum and/or diagnosing a disorder in the OCD spectrum by detecting in the subject a mutation in the SLITRK1 gene between the coding sequence for the second leucine rich repeat (LRR) domain and the transmembrane domain of the SLITRK1 protein. In another aspect, the invention further encompasses such a method wherein the mutation is a missense mutation. Such mutation may include a missense mutation resulting in a change at position 584 and/or position 593 of the SLITRK1 amino acid sequence of SEQ ID NO:1 or a corresponding position in other SLITRK1 proteins. Such mutation may further include a R→K mutation at position 584 and/or an S→G mutation at position 593 of the SLITRK1 amino acid sequence provided in SEQ ID NO:1 or a corresponding position in other SLITRK1 proteins. Such mutation may further include a G→A mutation at nucleotide 1751 and/or an A→G mutation at nucleotide 1777 of the SLITRK1 nucleotide sequence provided in SEQ ID NO:2 or a corresponding position in other SLITRK1 genes.

In another aspect, the invention encompasses a method of correlating a mutation in the SLITRK1 gene with an increased risk of developing a disorder in the OCD spectrum by detecting in a subject with the disorder the presence of one or more mutations in the SLITRK1 gene and correlating the presence of the one or more mutations in the SLITRK1 gene with the disorder in the subject. In particular embodiments, the disorder in the OCD spectrum is not Tourette's syndrome.

In another aspect, the invention encompasses a method of correlating a mutation in the SLITRK1 gene with a diagnosis of a disorder in the OCD spectrum by detecting in a subject diagnosed with the disorder the presence of one or more mutations in the SLITRK1 gene and correlating the presence of the one or more mutations in the SLITRK1 gene with the disorder in the subject. In particular embodiments, the disorder in the OCD spectrum is not Tourette's syndrome.

In another aspect, the invention encompasses a method of correlating a mutation in the SLITRK1 gene with an increased risk of developing a disorder in the OCD spectrum and/or a diagnosis of a disorder in the OCD spectrum by detecting in the subject a mutation in the SLITRK1 gene between the coding sequence for the second leucine rich repeat (LRR) domain and the transmembrane domain of the SLITRK1 protein. In another aspect, the invention further encompasses such a method wherein the mutation is a missense mutation.

In another aspect, the invention encompasses a method of correlating a mutation in the SLITRK1 gene with a good prognosis for a disorder in the OCD spectrum by detecting in a subject with the disorder and having a good prognosis, the presence of one or more mutations in the SLITRK1 gene and correlating the presence of the one or more mutations in the SLITRK1 gene with a good prognosis for the disorder.

In another aspect, the invention encompasses a method of identifying a subject with a disorder in the OCD spectrum as having a good prognosis by correlating the presence of the one or more mutations in the SLITRK1 gene with a good prognosis for the disorder and detecting the one or more mutations in the subject, thereby identifying the subject as having a good prognosis.

In another aspect, the invention encompasses a method of correlating a mutation in the SLITRK1 gene with a poor prognosis for a disorder in the OCD spectrum by detecting in a subject with the disorder and having a poor prognosis, the presence of one or more mutations in the SLITRK1 gene and correlating the presence of the one or more mutations in the SLITRK1 gene with a poor prognosis for the disorder.

In another aspect, the invention encompasses a method of identifying a subject with a disorder in the OCD spectrum as having a poor prognosis by correlating the presence of the one or more mutations in the SLITRK1 gene with a poor prognosis for the disorder and detecting the one or more mutations in the subject, thereby identifying the subject as having a poor prognosis.

In another aspect, the invention encompasses a method of correlating a mutation in the SLITRK1 gene with an effective treatment regimen for a disorder in the OCD spectrum by detecting in a subject with the disorder and for whom an effective treatment regimen has been identified, the presence of one or more mutations in the SLITRK1 gene and correlating the presence of the one or more mutations with an effective treatment regiment for the disorder.

In another aspect, the invention encompasses a method of identifying a treatment regimen for a subject with a disorder in the OCD spectrum by correlating the presence of one or more mutations in the SLITRK1 gene in a test subject with the disorder for whom an effective treatment regimen has been identified and detecting the one or more mutations in the subject, thereby identifying a treatment regimen for the subject.

In another aspect, the invention encompasses a method of correlating a mutation in the SLITRK1 gene with a prognosis and/or treatment regimen by detecting in the subject a mutation in the SLITRK1 gene between the coding sequence for the second leucine rich repeat (LRR) domain and the transmembrane domain of the SLITRK1 protein. In another aspect, the invention further encompasses a method wherein the mutation is a missense mutation. Such mutation may include a missense mutation resulting in an change at position 584 and/or position 593 of the SLITRK1 amino acid sequence of SEQ ID NO:1 or a corresponding position in other SLITRK1 proteins. Such mutation may further include a R→K mutation at position 584 and/or an S→G mutation at position 593 of the SLITRK1 amino acid sequence provided in SEQ ID NO:1 or a corresponding position in other SLITRK1 proteins. Such mutation may further include a G→A mutation at nucleotide 1751 and/or an A→G mutation at nucleotide 1777 of the SLITRK1 nucleotide sequence provided in SEQ ID NO:2 or a corresponding position in other SLITRK1 genes.

The invention encompasses the methods disclosed herein where the disorder is selected from the group consisting of bulimia, anorexia nervosa, anxiety, depression, skin picking, motor tics, trichotillomania, and any combination thereof

The invention encompasses the methods disclosed which can comprise detecting a mutation at the nucleic acid (DNA or RNA) level and/or at the amino acid level.

In another aspect, the invention encompasses a method of identifying a candidate compound for the treatment of a disorder in the OCD spectrum by contacting a SLITRK1 protein or functional portion thereof with a test compound and detecting binding of the compound to the SLITRK1 protein and/or modulation of the SLITRK1 protein activity where a compound that binds to the SLITRK1 protein and/or modulates SLITRK1 protein activity as compared with the activity in the absence of the test compound is identified as a candidate compound for the treatment of the disorder. In another aspect, the invention further encompasses such a method wherein the SLITRK1 protein comprises a mutation associated with the disorder. In another aspect, the invention further encompasses such a method wherein the SLITRK1 protein does not comprise a mutation associated with the disorder, and optionally is a wild-type SLITRK1 protein.

In another aspect, the invention encompasses a method of identifying a candidate compound for the treatment of a disorder in the OCD spectrum by contacting a SLITRK1 nucleic acid or functional portion thereof with a test compound and detecting binding of the compound to the SLITRK1 nucleic acid and/or modulation of the SLITRK1 nucleic acid activity, wherein a compound that binds to the SLITRK1 nucleic acid and/or modulates SLITRK1 nucleic acid activity as compared with the activity in the absence of the test compound is identified as a candidate compound for the treatment of the disorder. In another aspect, the invention further encompasses such a method wherein the SLITRK1 nucleic acid comprises a mutation associated with the disorder. In another aspect, the invention further encompasses such a method wherein the SLITRK1 nucleic acid does not comprise a mutation associated with the disorder, and optionally is a wild-type SLITRK1 nucleic acid.

### DESCRIPTION OF THE DRAWINGS

FIG. 1 shows the amino acid sequence of a human SLITRK1 (SEQ ID NO:1). In the amino acid sequence schematic, the first and second putative leucine rich repeat (LLR) domains are labeled as such and the sequence of both is boxed and bolded. The transmembrane region is labeled as such and the sequence is bolded and underlined. The sites of the two TMM mutations are labeled as such and the two amino acids are boxed and shaded.

FIG. 2 shows the mRNA sequence of a human SLITRK1 (SEQ ID NO:2, NCBI Accession No. NM_{—}052910, incorporated by reference herein in its entirety). The sequence comprises the upstream 5′ noncoding region (nucleotides 1-886) as well as the protein-coding region (beginning at nucleotide 887). The start codon (ATG) is underlined, bolded, and capitalized. The sites of the two TMM mutations are boxed and shaded.

FIG. 3 shows the genetic organization of the SLITRK1 mRNA sequence. The schematic shows the two new detected mutations S593G and R584K in trichotillomania. Also shown are the L422fsˆ deletion and c.*689G>Aˆˆ var321 previously reported in connection with Tourette's syndrome. Bars along the lower bottom represent conserved protein domains according to CCDB (LRR—leucine-rich repeat; LRRCT—leucine-rich repeat C-terminal domain; TM—transmembrane domain; miR—189—micro RNA binding site).

FIG. 4 shows the detected mutations in trichotillomania families DUK14044 (SEQ ID NOS:3 and 4) and DUK14002 (SEQ ID NOS: 5 and 6). The pedigrees of the identified families and sequencing traces of controls and affected subjects are shown. Arrows indicate the index patients. Black symbols are trichotillomania; open symbols are unaffected; shaded symbol is mood disorder/low self-esteem.

FIG. 5 shows a fragment of the corresponding amino acid sequence from homologs of the human SLITRK1 protein (SEQ ID NO:7) in mouse (SEQ ID NO:8), cow (SEQ ID NO:9), dog (SEQ ID NO:10) and pufferfish (SEQ ID NO:11). The two novel mutations are highly conserved across different species. Dark grey shading represents the identified mutations; light grey shading represents the conserved residues.

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Definitions

As used herein, “a,” “an” or “the” can mean one or more than one. For example, “a” mutation can mean a single mutation or a multiplicity of mutations.

As used herein, “and/or” refers to and encompasses any and all possible combinations of one or more of the associated listed items, as well as the lack of combinations when interpreted in the alternative (“or”).

As used herein, the term “disorder in the OCD spectrum” (and similar terms) includes any disorder or condition that is associated with obsessive compulsive disorder (OCD). See, e.g., Diagnostic and Statistical Manual of Mental Disorders—Fourth Edition (DSM-IV). Illustrative disorders in the OCD spectrum include but are not limited to eating disorders (e.g., bulimia and/or anorexia nervosa), anxiety (e.g., social anxiety), depression, skin picking, motor tics, trichotillomania (TTM), and any combination thereof In representative embodiments, the term “disorder in the OCD spectrum” excludes Tourette's syndrome (TS). In particular embodiments, the disorder is TTM not associated with TS.

Subjects according to the present invention include humans as well as animal models (e.g., mammalian models) of human psychiatric disorders. Suitable subjects include without limitation non-human primates, dogs, cats, horses, cattle, pigs, sheep, goats, guinea pigs, mice, rats, rabbits and chickens. The subject can be homozygous or heterozygous for the mutations described herein.

As used herein, the term “nucleic acid” encompasses both RNA and DNA, including cDNA, genomic DNA, mRNA, synthetic (e.g., chemically synthesized) DNA and chimeras of RNA and DNA. The nucleic acid can be double-stranded or single-stranded. Where single-stranded, the nucleic acid can be a sense strand or an antisense strand. The nucleic acid can be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

As used herein, the term “gene” refers to a segment of nucleic acid that contains the information necessary to produce a functional RNA product. A gene usually contains regulatory regions dictating under what conditions the RNA product is made, transcribed regions dictating the sequence of the RNA product, and/or other functional sequence regions. A gene may be transcribed to produce an mRNA molecule, which contains the information necessary for translation into the amino acid sequence of the resulting protein.

As used herein, the term “coding sequence” or “protein-coding sequence” refers to the region of a gene and/or mRNA sequence that encodes the amino acid sequence of the resulting protein.

As used herein, the term “wild-type” refers to the typical sequence or sequences of a gene and/or protein in nature, i.e. the most common sequence or sequences in the natural population. This may, however, over a period of time be replaced by another form and/or vary between populations within the same species.

An “isolated nucleic acid” is a nucleic acid that is not immediately contiguous with nucleotide sequences with which it is immediately contiguous (one on the 5′ end and one on the 3′ end) in the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about 6, 8, 10, 12, 15, 20, 25 or 30 nucleotides and/or less than about 20, 25, 30, 35, 50, 60, 75 or 100 nucleotides or more, for example, about 12 to 60 nucleotides, or about 15 to 35 nucleotides, which can be used, for example, as a primer in an amplification assay or as a probe in a hybridization assay or in a microarray. Oligonucleotides can be natural or synthetic, e.g., DNA, RNA, modified backbones, etc.

The term “mutation” refers to one or more changes to the sequence of a DNA sequence or a protein amino acid sequence relative to a reference sequence, usually a wild-type sequence. A mutation in a DNA sequence may or may not result in a corresponding change to the amino acid sequence of the encoded protein. A mutation may be a point mutation, i.e. an exchange of a single nucleotide and/or amino acid for another. Point mutations that occur within the protein-coding region of a gene's DNA sequence may be classified as a silent mutation (coding for the same amino acid), a missense mutation (coding for a different amino acid), and a nonsense mutation (coding for a stop which can truncate the protein). A mutation may also be an insertion, i.e. an addition of one or more extra nucleotides and/or amino acids into the sequence. Insertions in the coding region of a gene may alter splicing of the mRNA (splice site mutation), or cause a shift in the reading frame (frameshift), both of which can significantly alter the gene product. A mutation may also be a deletion, i.e. removal of one or more nucleotides and/or amino acids from the sequence. Deletions in the coding region of a gene may alter the splicing and/or reading frame of the gene. A mutation may be spontaneous, induced, naturally occurring, or genetically engineered.

As used herein, detecting a mutation in a subject may be done by any method useful for analyzing the DNA or amino acid sequence of the subject for the presence or absence of a mutation. Such methods for analyzing a DNA or amino acid sequence are well known to those of skill in the art and any suitable means of detecting a mutation are encompassed by the present invention. Such analysis may be done, for example, by isolating a genomic DNA sample from the subject and using nucleic acid hybridization with a detectable probe to test for the presence and/or absence of a mutation. Alternately, such analysis may be done using an mRNA sample from the subject, and optionally producing cDNA from the sample. Such analysis may also be done, for example, using polymerase chain reaction to amplify a nucleic acid sequence and the amplification product may be sequenced and/or used for hybridization with a probe to detect the mutation. Such analysis may also be done, for example, by isolating a protein sample from the subject and using antibodies to test for the presence and/or absence of a mutation in the protein.

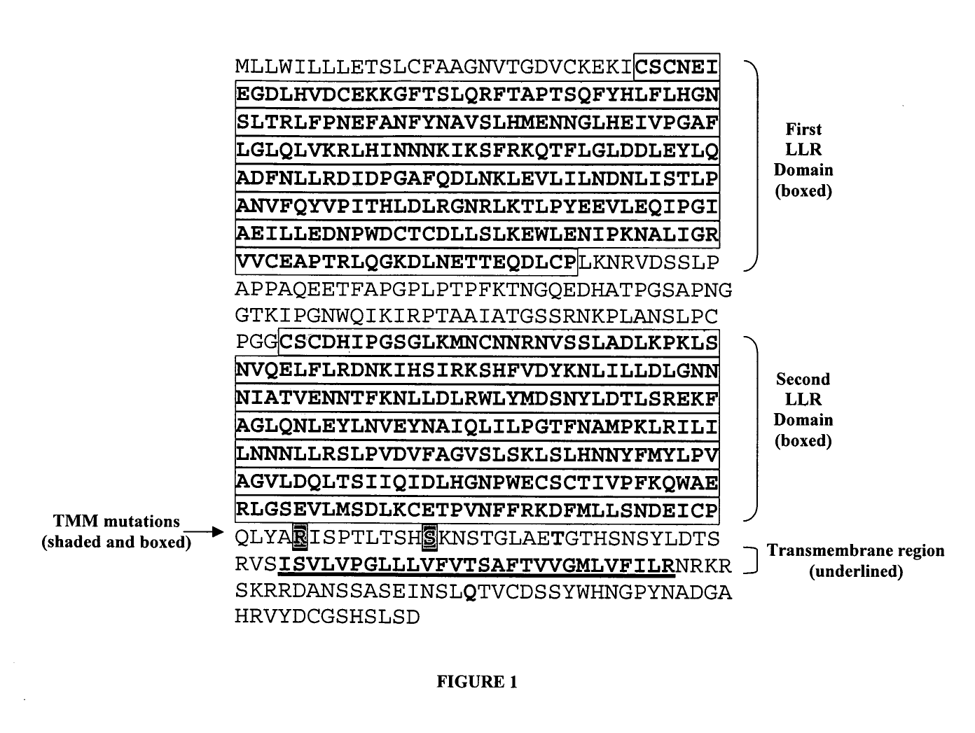

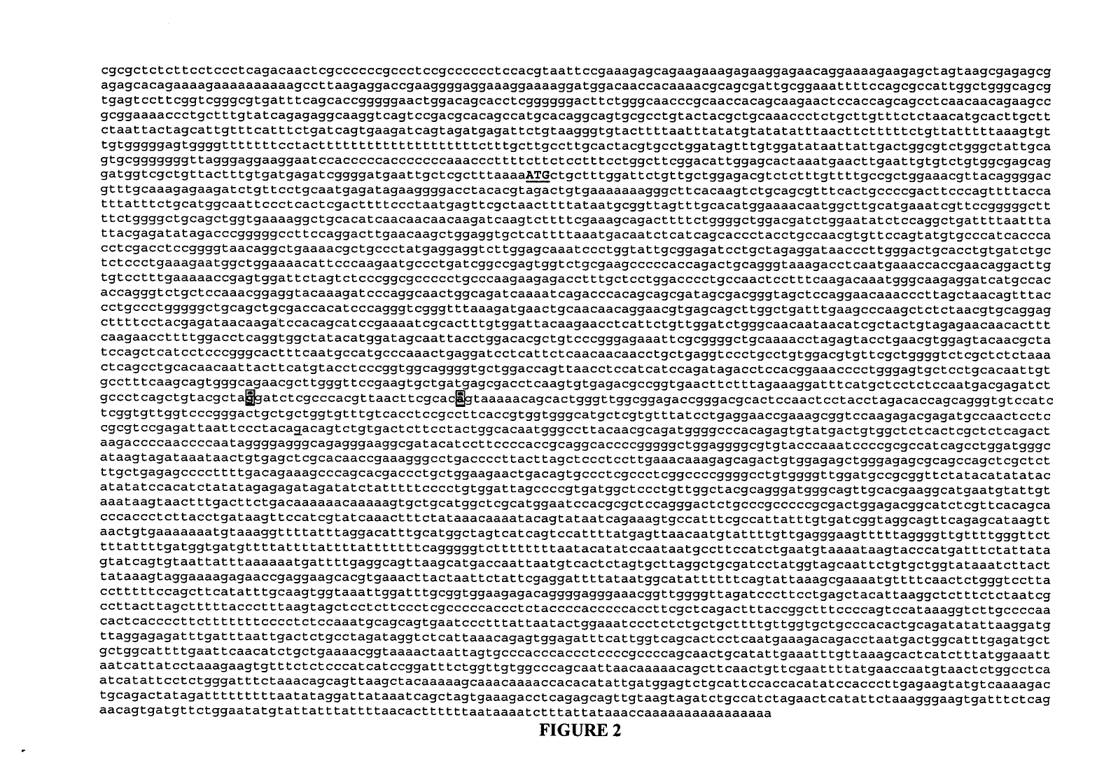

The present invention is based, in part, on the inventors' discovery of a correlation between two mutations in the SLITRK1 gene and disorders in the OCD spectrum. Thus, as one aspect, the present invention provides a method of identifying a subject having an increased risk of developing a disorder in the OCD spectrum, the method comprising detecting in the subject a mutation in the SLITRK1 gene associated with an OCD spectrum disorder. In particular embodiments, the mutation is located between the coding sequence for the second leucine rich repeat (LRR) domain and the transmembrane domain of the SLITRK1 protein (see FIG. 1 which illustrates this in the amino acid sequence and FIG. 2 which illustrates this in the nucleic acid sequence). See also NCBI Accession No. NM_{—}052910.

The invention further provides a method of diagnosing a disorder in the OCD spectrum in a subject, the method comprising detecting in the subject a mutation in the SLITRK1 gene associated with the disorder. In particular embodiments, the mutation is located between the coding sequence for the second LRR domain and the transmembrane domain of the SLITRK1 protein (see FIGS. 1 and 2).

The methods of the invention can be used alone or in combination with other diagnostic tools to confirm a diagnosis, e.g., diagnostic tools based on behavior.

OCD spectrum disorders are as described above.

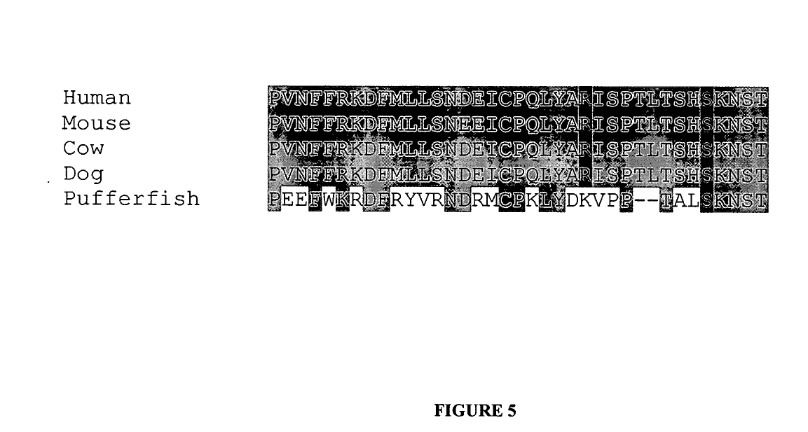

In representative embodiments, the mutation is located between amino acid positions 580 and 616 (inclusive) of the human SLITRK1 sequence of SEQ ID NO:1 (see also FIG. 1) or the corresponding positions of other SLITRK1 proteins. This region is evolutionarily conserved from Tetraodon nigroviridis (green spotted pufferfish) to humans (see FIG. 5). In particular embodiments, the mutation is located between amino acid 580, 582 or 584 and amino acid 593, 600, 602, 604, 606, 608, 610, 612, 614 or 616 (inclusive) of the SLITRK1 sequence of SEQ ID NO:1 (see also FIG. 1) or the corresponding position of other SLITRK1 proteins, including any combination of lower and upper limits. Those skilled in the art will appreciate that other SLITRK1 proteins and/or other SLITRK1 genes are known in the art and/or may be readily identified by one of ordinary skill in the art using established methods. For example, SLITRK1 variants and SLITRK1 homologs may exist among humans as well as among members of other species and may vary in sequence from the human sequence of SEQ ID NO:1 (see also FIG. 1). Nonetheless, those skilled in the art can routinely identify amino acid positions in such variants and homologs “corresponding to” the specified positions in the amino acid and nucleic acid sequences of SEQ ID NO:1 and SEQ ID NO:2 (see also FIGS. 1 and 2) (e.g., with alignment algorithms).

The mutation can be any mutation known in the art including without limitation missense mutations, nonsense mutations, insertions, deletions, frameshift mutations, single nucleotide polymorphisms, and the like. Generally, the mutation is not a silent mutation. In particular embodiments, the mutation comprises, consists essentially of, or consists of a missense mutation. The missense mutation can be a point mutation (i.e., resulting in the change of one amino acid) or can result in a change in two or more (e.g., two, three, four, five, six, etc.) amino acids in the encoded protein.

By “consisting essentially of” is meant that the mutation does not comprise any material element beyond the specified elements.

In particular embodiments, the mutation comprises, consists essentially of or consists of a missense mutation resulting in a change at position 584 and/or position 593 of the SLITRK1 amino acid sequence of SEQ ID NO:1 (see also FIG. 1) or a corresponding position in other SLITRK1 proteins.

In representative embodiments, the mutation comprises, consists essentially of or consists of a R→K, Y or T mutation at position 584 and/or an S→G, T, Y, P, Q, A, V, L or I mutation at position 593 of the SLITRK1 amino acid sequence provided in SEQ ID NO:1 (see also FIG. 1) or a corresponding position in other SLITRK1 proteins.

Optionally, the missense mutation results from a G→A mutation at nucleotide 1751 of the SLITRK1 coding sequence (corresponding nucleotide 2637 in SEQ ID NO:2) and/or an A→G mutation at nucleotide 1777 of the SLITRK1 coding sequence (corresponding nucleotide 2663 in SEQ ID NO:2) provided in FIG. 2 (wherein the ATG start codon is underlined marking the beginning of the SLITRK1 coding sequence) or a corresponding position in other SLITRK1 genes.

In particular embodiments, SLITRK1 mutations according to the present invention exclude the L422fsˆ deletion and c.*689G>Aˆˆ var321 mutations previously described by J F Abelson, et al. Science. 2005 310(5746):317-20.

Other SLITRK1 genes and proteins, i.e. SLITRK1 homologues, may be useful in the practice of some embodiments of the present invention and can be identified be any means known to those skilled in the art. Such means may include, for example, searching a database of sequences using SEQ ID NO:1 or SEQ ID NO:2 to identify sequences having high sequence identity to SEQ ID NO:1 or SEQ ID NO:2. Such identified sequences may be considered SLITRK1 homologues and would be useful for practicing the methods of the present invention.

Different nucleic acids or proteins having homology are referred to herein as “homologues”. The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. For example, FIG. 5 illustrates a sequence alignment of corresponding amino acid sequence regions of homologues of the human SLITRK1 protein in different species. Mutations in corresponding conserved regions of SLITRK1 homologues are encompassed by the methods of the present invention.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGO Wisconsin Package®. (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™. (Version 10; Genetics Computer Group, Inc., Madison, Wis.). “Gap” utilizes the algorithm of Needleman and Wunsch (Needleman and Wunsch, Journal of Molecular Biology 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983).

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

The invention also provides a method of identifying a subject having an increased risk of developing a disorder in the OCD spectrum, the method comprising:

(a) correlating the presence of one or more mutations in the SLITRK1 gene with an increased risk of developing the disorder; and (b) detecting the one or more mutations of step (a) in the subject, thereby identifying the subject as having an increased risk of developing the disorder.

Correlating the presence of one or more mutations with an increased risk may be done by identifying the presence of a mutation in a subject and associating this presence in the subject with a disorder in the OCD spectrum, or vice versa. Such correlation may be done with one or more subjects.

Also provided is a method of diagnosing a disorder in the OCD spectrum in a subject, the method comprising: (a) correlating the presence of one or more mutations in the SLITRK1 gene with an increased risk of developing the disorder; and (b) detecting the one or more mutations of step (a) in the subject, thereby diagnosing the subject as having the disorder.

OCD spectrum disorders and SLITRK1 mutations are as described above.

The invention also provides a method of correlating a mutation in the SLITRK1 gene with an increased risk of developing a disorder in the OCD spectrum, the method comprising: (a) detecting in a subject with the disorder the presence of one or more mutations in the SLITRK1 gene; and (b) correlating the presence of the one or more mutations in the SLITRK1 gene of step (a) with the disorder in the subject.

Also provided is a method of correlating a mutation in the SLITRK1 gene with a diagnosis of a disorder in the OCD spectrum, the method comprising: (a) detecting in a subject diagnosed with the disorder the presence of one or more mutations in the SLITRK1 gene; and (b) correlating the presence of the one or more mutations in the SLITRK1 gene of step (a) with the disorder in the subject.

OCD spectrum disorders and SLITRK1 mutations are as described above.

The invention also provides methods of determining whether a subject will have a good or poor prognosis for a disorder in the OCD spectrum. Thus, in particular embodiments, the invention provides a method of correlating a mutation in the SLITRK1 gene with a good or poor prognosis for a disorder in the OCD spectrum, the method comprising: (a) detecting in a subject with the disorder and having a good or poor prognosis, the presence of one or more mutations in the SLITRK1 gene; and (b) correlating the presence of the one or more mutations in the SLITRK1 gene of step (a) with a good or poor prognosis, respectively, for the disorder.

In further embodiments, the invention provides a method of identifying a subject with a disorder in the OCD spectrum as having a good or poor prognosis, the method comprising: (a) correlating the presence of the one or more mutations in the SLITRK1 gene with a good or poor prognosis for the disorder; and (b) detecting the one or more mutations of step (a) in the subject, thereby identifying the subject as having a good or poor prognosis, respectively.

The invention also facilitates the development of personalized therapies based on the underlying genetic cause of an OCD spectrum disorder. Patients who respond well to particular treatment protocols can be analyzed for specific mutations and a correlation can be established according to the methods provided herein. Alternatively, patients who respond poorly to a particular treatment regimen can also be analyzed for particular mutations correlated with the poor response. Then, a subject who is a candidate for treatment for an OCD spectrum disorder can be assessed for the presence of the appropriate mutation(s) and a targeted treatment regimen can be provided.

Thus, in particular embodiments, the invention provides a method of correlating a mutation in the SLITRK1 gene with an effective treatment regimen for a disorder in the OCD spectrum, the method comprising: (a) detecting in a subject with the disorder and for whom an effective treatment regimen has been identified, the presence of one or more mutations in the SLITRK1 gene; and (b) correlating the presence of the one or more mutations of step (a) with an effective treatment regimen for the disorder.

The invention further provides a method of identifying a treatment regimen for a subject with a disorder in the OCD spectrum, the method comprising: (a) correlating the presence of one or more mutations in the SLITRK1 gene in a test subject with the disorder for whom an effective treatment regimen has been identified; and (b) detecting the one or more mutations of step (a) in the subject, thereby identifying a treatment regimen for the subject.

OCD spectrum disorders and SLITRK1 mutations are as described above.

Treatment regimens for disorders in the OCD spectrum are known in the art and include but are not limited to behavioral therapy, cognitive therapy, psychotherapy, surgery, medications, or any combination of these. Medications and compounds useful in treating disorders in the OCD spectrum include, but are not limited to, selective serotonin reuptake inhibitors (SSRIs) such as paroxetine (Paxil®, Aropax®), sertraline (Zoloft®), fluoxetine (Prozac®), and fluvoxamine (Luvox®, Faverin®, Fevarin® and Dumyroxg); gabapentin (Neurontin®); lamotrigine (Lamictal®); atypical antipsychotics such as olanzapine (Zyprexa®) and risperidone (Risperdal®); the sugar inositol; opioids such as tramadol (Ultram®) and hydrocodone (Vicodin®); nicotine; hallucinogens such as tryptamine alkaloid psilocybin, LSD, and peyote; tricyclic antidepressants such as clomipramine (Anafranil®); and any combination thereof.

The invention further provides isolated SLITRK1 proteins and nucleic acids (e.g., genomic nucleic acid or cDNA) and fragments thereof comprising the mutations described herein.

Methods of detecting mutations at the nucleic acid or amino acid level are also well known in the art and can be used in the practice of the present invention.

For example, DNA can be obtained from any suitable sample from the subject that contains DNA, and the DNA can be prepared and analyzed according to well-established protocols for the presence of the mutations described herein. In some embodiments, analysis of the DNA can be carried out by amplification of the region of interest according to amplification protocols well-known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA)). The amplification product can then be visualized directly in a gel by staining or the product can be detected by hybridization with a detectable probe. Optionally, the amplification product is sequenced to detect the mutation.

Amplification primers and probes can be selected to be specific for the mutated gene or amplify/detect both wild-type and mutated sequences. When amplification conditions or primer location allows for amplification of both wild-type and mutated genes, they can be distinguished by a variety of well-known methods, such as hybridization with a mutant-specific probe, secondary amplification with mutant-specific primers, by restriction endonuclease digestion, or by electrophoresis.

The present invention further provides fragments or oligonucleotides, which can be used as primers and/or probes for detecting and/or identifying the mutations described herein. Thus, in some embodiments, a fragment or oligonucleotide of this invention is a nucleotide sequence that is at least about 10, 15, 20, 25, 30, 35, 40, 45, 50, 55, 60, 65, 70, 75, 80, 85, 90 or 100 and/or less than about 25, 35, 50, 75, 100, 125, 150, 175, 200, 250, 300, 350, 400, 450, 500, 550, 600, 650, 700, 750, 800, 850, 900, 1000, 1500, 2000, 2500 or 3000 contiguous nucleotides of the nucleotide sequence set forth in SEQ ID NO:2 as modified to comprise a mutation described herein. In particular embodiments, the fragment or oligonucleotide spans one or both of the mutation sites shown in FIGS. 1 and/or 2 and further comprises a T (or U) at the position corresponding to nucleotide 1751 of the coding sequence shown in FIG. 2 and/or comprises a C at the position corresponding to nucleotide 1777 of the coding sequence shown in FIG. 2. Such fragments or oligonucleotides can be detectably labeled or modified, for example, to include and/or incorporate a restriction enzyme cleavage site when employed as a primer in an amplification (e.g., PCR) assay.

Mutations in the SLITRK1 gene described herein can be correlated with OCD spectrum disorders according to methods well known in the art and as disclosed herein. In general, identifying such a correlation involves conducting analyses that establish a statistically significant association and/or a statistically significant correlation between the presence of a genetic marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed.

The correlation can involve one or more than one (e.g., two, three, four or more) mutations in the SLITRK1 gene or a combination of one or more mutations in the SLITRK1 gene and one or more mutations at other loci.

In some embodiments, the methods of correlating a mutation(s) with treatment regimens can be carried out using a computer database. Thus, the present invention provides a computer-assisted method of identifying a proposed treatment for an OCD spectrum disorder. The method comprises the steps of (a) storing a database of biological data for a plurality of patients, the biological data that is being stored including for each of said plurality of patients (i) a treatment type, (ii) at least one SLITRK1 mutation associated with an OCD spectrum disorder, and (iii) at least one disease progression measure for the disorder from which treatment efficacy can be determined; and then (b) querying the database to determine the association of said mutation with the effectiveness of a treatment protocol in treating the OCD spectrum disorder, to thereby identify a proposed treatment as a treatment (e.g., an effective treatment) for a subject carrying a particular SLITRK1 mutation correlated with the OCD spectrum disorder.

In one embodiment, treatment information for a patient is entered into the database (through any suitable means such as a window or text interface), SLITRK1 mutation information for that patient is entered into the database, and disease progression information is entered into the database. These steps are then repeated until the desired number of patients has been entered into the database. The database can then be queried to determine whether a particular treatment is effective for patients carrying a particular marker, not effective for patients carrying a particular marker, etc. Such querying can be carried out prospectively or retrospectively on the database by any suitable means, but is generally done by statistical analysis in accordance with known techniques.

The discovery of the connection between SLITRK1 and OCD spectrum disorders provides the basis for drug discovery methods using a SLITRK1 nucleic acid (e.g., genomic or cDNA) and/or protein and/or pathways associated with SLITRK1 protein function as a target. For example, the SLITRK1 nucleic acid and/or protein (or functional portions of either of the foregoing) can be used as a target in methods of screening for compounds, including high throughput methods, that bind to and/or modulate (e.g., decrease or increase) the activity of the SLITRK1 nucleic acid and/or protein as candidate compounds for the treatment of an OCD spectrum disorder.

In representative embodiments, the SLITRK1 promoter is operatively associated with a reporter gene (e.g., LacZ, β-galactosidase) and introduced into a cultured cell or cell in vivo and used to screen compounds that modulate SLITRK1 promoter activity.

Compounds that modulate SLITRK1 nucleic acid or protein activity can act at the transcriptional, post-transcriptional, translational and/or post-translational level, the latter including post-translational modifications and protein turnover. The compound can modulate the amount of the protein and/or nucleic acid and/or modulate the activity of the protein and/or nucleic acid.

Test compounds that can be screened in accordance with the methods provided herein encompass numerous chemical classes including, but not limited to, synthetic or semi-synthetic chemicals, purified natural products, proteins, antibodies, peptides, peptide aptamers, nucleic acids, oligonucleotides (e.g., including antisense and RNAi such as shRNA and siRNA), carbohydrates, lipids, or other small or large organic or inorganic molecules. Small molecules are desirable because such molecules are more readily absorbed after oral administration and have fewer potential antigenic determinants. Non-peptide agents or small molecule libraries are generally prepared by a synthetic approach, but recent advances in biosynthetic methods using enzymes may enable one to prepare chemical libraries that are otherwise difficult to synthesize chemically.

Small molecule libraries can be obtained from various commercial entities, for example, SPECS and BioSPEC B. V. (Rijswijk, the Netherlands), Chembridge Corporation (San Diego, Calif.), Comgenex USA Inc., (Princeton, N.J.), Maybridge Chemical Ltd. (Cornwall, UK), and Asinex (Moscow, Russia). One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, e.g., Tan et al., (1998) Am. Chem Soc. 120: 8565-8566; and Floyd et al., (1999) Prog Med Chem 36:91-168. Other commercially available libraries can be obtained, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. In certain embodiments of the invention the methods are performed in a high-throughput format using techniques that are well known in the art, e.g., in multiwell plates, using robotics for sample preparation and dispensing, etc. Representative examples of various screening methods may be found, for example, in U.S. Pat. Nos. 5,985,829; 5,726,025; 5,972,621; and 6,015,692. The skilled practitioner will readily be able to modify and adapt these methods as appropriate.

Thus, in particular embodiments, the invention provides a method of identifying a candidate compound for the treatment of an OCD spectrum disorder, the method comprising contacting a SLITRK1 nucleic acid or protein (or functional portion of either of the foregoing) with a test compound, detecting binding of the compound to the SLITRK1 nucleic acid or protein and/or modulation of the SLITRK1 nucleic acid or protein activity, wherein a compound that binds to the SLITRK1 nucleic acid or protein and/or modulates SLITRK1 nucleic acid or protein activity (e.g., as compared with the activity in the absence of the test compound) is identified as a candidate compound for the treatment of the disorder.

The term “binding” refers to a chemical interaction between molecules. Binding may be between a test compound and a SLITRK1 nucleic acid or between a test compound and a SLITRK1 protein. Such binding may be detected by any suitable method known in the art including without limitation blot analysis, Fluorescent-activated cell sorting (FACS), Enzyme-Linked Immunosorbent Assay (ELISA), affinity column analysis, Fluorescence resonance energy transfer (FRET), and any method in vivo and/or in vitro for assessing loss or gain of function and/or activity as a result of contact with a test compound. Such techniques are well known to those skilled in the art. See, e.g., J. Sambrook & D. Russell, Molecular Cloning: A Laboratory Manual 3rd Ed. (2001) (Cold Spring Harbor Laboratory Press; Woodbury, N.Y.) and Current Protocols In Molecular Biology, edited by F. M. Ausubel et al. (John Wiley & Sons, Inc.; Hoboken, N.J.); and Current Protocols in Cell Biology, edited by Juan S. Bonifacino, et al. (John Wiley & Sons, Inc.; Hoboken, N.J.).

SLITRK1 protein activity can be assessed by any suitable method known in the art including without limitation measuring binding to proteins and/or nucleic acids, measuring biological activity (e.g., promotion of neurite outgrowth and/or establishment of neural connections and/or establishment of neural patterning) or the activity of another protein or nucleic acid in a pathway regulated by SLITRK1, or any other activity known in the art.

A “functional” portion of a SLITRK1 protein is a portion that retains one or more SLITRK1 protein activities (e.g., 50%, 60%, 70%, 80%, 90%, 95%, 97% or 100% or more of the activity of a full-length SLITRK1 protein). A “functional” portion of a SLITRK1 nucleic acid encodes a functional portion of a SLITRK1 protein.

SLITRK1 nucleic acid activity can also be assessed by any method known in the art, including but not limited to transcriptional activity, post-transcriptional activity, and protein binding.

Screening assays can be cell-free, cell-based or animal based. Further, the SLITRK1 nucleic acid or protein can comprise a mutation as described herein or can be a SLITRK1 nucleic acid or protein that is not associated with an OCD spectrum disorder (for example, a wild-type nucleic acid or protein, e.g., the sequences shown in FIGS. 1 and 2).

Genetically modified animals comprising a SLITRK1 nucleic acid comprising a mutation as described herein can be used for in vivo screening methods to identify compounds that bind to and/or modulate SLITRK1 nucleic acid and/or modulate protein activity and/or to identify compounds that modulate the phenotype (e.g., behaviors) associated with the mutation including but not limited to behaviors associated with TTM (e.g., hair pulling), skin picking, motor tics, anxiety, depression and/or eating disorders. Methods of making genetically modified animals are known in the art. Suitable animals include any non-human mammal including without limitation laboratory animals (e.g., mice, rats, hamsters, rabbits, guinea pigs, etc.), pigs, cattle, goats, sheep, cats and dogs.

The invention further provides antibodies that recognize the mutant SLITRK1 proteins described herein. Optionally, the antibody does not recognize the wild-type form of the SLITRK1 protein (see, e.g., SEQ ID NO:1). Such antibodies can be used in the screening and diagnostic methods of the invention and can further be used as a research reagent to purify and/or study the function of SLITRK1.

The term “antibody” refers to an immunoglobulin molecule (including IgG, IgE, IgA, IgM, IgD) and/or immunologically active portions of immunoglobulin molecules, i.e., molecules that contain an antibody combining site or paratope and can bind antigen. An “antibody combining site” or “antigen binding site” is that structural portion of an antibody molecule comprised of heavy and light chain variable and hypervariable (CDR) regions that specifically binds antigen. As is known in the art, particular properties of antibodies relate to immunoglobulin.isotype. The antibody or fragment can be monoclonal or polyclonal. The antibody or fragment can further be from any species of origin including avian (e.g., chicken, turkey, duck, geese, quail, etc.) and mammalian (e.g., human, non-human primate, mouse, rat, rabbit, cattle, goat, sheep, horse, pig, dog, cat, etc.) species.

As yet another aspect, the invention provides methods of administering to an animal comprising a SLITRK1 mutation associated with an OCD spectrum disorder, a nucleic acid encoding a SLITRK1 protein (or functional portion thereof) that does not comprise a mutation associated with an OCD spectrum disorder (e.g., a wild-type SLITRK1 nucleic acid). Alternatively (or additionally), the invention provides a method of delivering an interfering RNA (e.g., siRNA or shRNA) or an antisense RNA directed against a mutant SLITRK1 associated with an OCD spectrum disorder to reduce expression of the mutant SLITRK1. Methods of delivering a functional SLITRK1 nucleic acid or knocking down expression of a mutant SLITRK1 can be used in a research setting to study the function of SLITRK1 and/or as a therapeutic method in a subject in need thereof (e.g., a subject comprising a mutant SLITRK1 associated with an OCD spectrum disorder). Suitable animal subjects according to this embodiment of the invention are as described above for diagnostic screening methods.

### EXAMPLES

Sequence variation in the SLITRK1 gene correlates with disorders in the OCD spectrum, such as TTM not associated with TS. We identified two non-synonomous mutations within a region of the SLITRK1 gene between the second leucine rich repeat (LRR) domain and the transmembrane domain in two unrelated patients with TTM. In one patient, TMM was co-morbid with skin picking, motor tics, mild anxiety, and a history of depression. In both cases, a carrier parent was identified who also exhibited psychiatric difficulties. These mutations were not identified in over 2,000 control subjects.

We ascertained 44 TTM families in which one or more individuals met DMS-IV criteria for TTM. Self-reports of frequency and duration of hair pulling, related psychiatric symptoms, family history, and blood were obtained from patients and family members under IRB-approved procedures by Duke University Medical Center.

We then directly re-sequenced the complete SLITRK1 gene in the index cases of 44 TTM families. Overlapping PCR products covering the entire sequence of the SLITRK1 gene were directly sequenced by applying the ABI Prism® BigDye™ Terminator Cycle Sequencing Kit (Applied Biosystems, Foster City, Calif. USA), and then subjected to an Applied Biosystems 3730 DNA Analyzer.

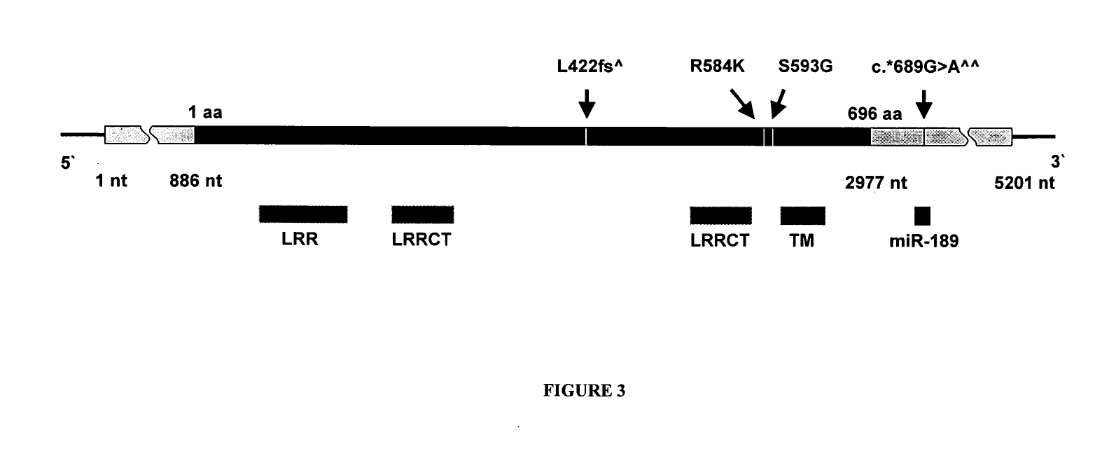

We identified two novel non-synonymous sequence changes that occurred in two independent TTM subjects of European descent. FIG. 3 illustrates the genetic organization of the SLITRK1 gene. FIG. 3 shows the two sequence changes 5593G and R584K in TTM. FIG. 3 also shows the reported changes L422FSˆ deletion and c.*689G>AAˆˆ321 in Tourette's syndrome. Bars along the lower bottom of FIG. 3 represent conserved protein domains according to CCDB (LRR - leucine-rich repeat; LRRCT—leucine-rich repeat C-terminal domain; TM—transmembrane domain; miR—189—micro RNA binding site). FIG. 5 is an alignment of the relevant amino acid sequence for these two novel mutations across different species, demonstrating that they are highly conserved. In FIG. 5 dark grey shading represents the identified mutations and light grey shading represents the conserved residues.

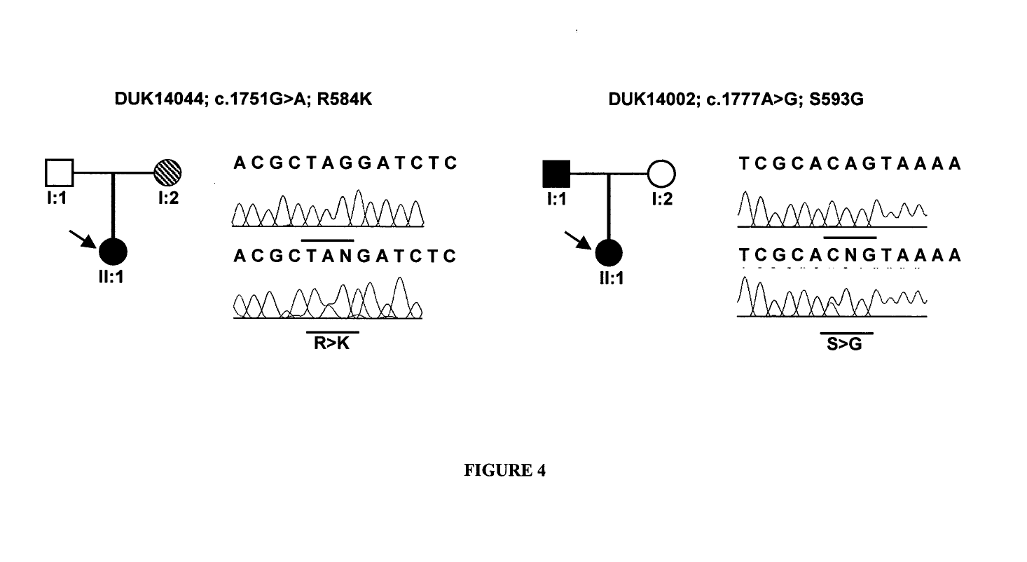

In family DUK14044 a G>A transition resulted in substitution of arginine for lysine; c.1751G>A, R584K In a second family (DUK14002), only nine residues downstream, a serine was replaced by glycine; c. 1 777A>G, S593G. FIG. 4 shows the detected mutations in trichotillomania families DUK14044 and DUK14002. The pedigrees of the identified families and sequencing traces of controls and affected subjects are shown. Arrows indicate the index patients. Black symbols are trichotillomania; open symbols are unaffected; shaded symbol is mood disorder/low self-esteem. These sequence variations were not detected in the other TTM cases. Both changes occurred within a short distance between the second LRRCT domain and the transmembrane domain of SLITRK1 (FIG. 3). Mutations in this position may be associated with TTM in the absence of TS since the mutation previously reported was in a different part of the protein. (JF Abelson, et al. Science. 2005 310(5746):317-20.)

We then designed Taqman genotyping assays and screened for the presence of these variations in a sample of 2,192 non-TTM Caucasian controls. We controlled for the presence of these variations by using the two TTM patients as positive controls for the assays. We did not detect the altered genotypes in this sample. Thus, these sequence changes are strongly associated with the TTM phenotype (p=0.0004, Fisher's exact test).

Individual II:1 of family DUK14044 was 38 years old and reported hair pulling since the age of nine (FIG. 4). She also picked at her skin and presented with motor tics. Although she had a history of depression, current self-reported mood on the Beck Depression Inventory was within normal limits. She had mild anxiety as evidenced by a slight elevation on the State Trait Anxiety Inventory. Her father (I:1) had no history of psychiatric disorder. Her mother (I:2), who is a mutation carrier, had a history of depression, low self-esteem, and fear of heights. However, there was no evidence of TTM.

The index case of family DUK14002 (II: 1) was diagnosed with TTM at the age of nine (FIG. 4). Currently, at the age of 14, her TTM symptoms have remitted but she twirls her hair and bites her nails. Based on self- and parent-report, there were no clinically significant mood, anxiety, or behavior problems. Her father, who was 46 years of age, was the mutation carrier. He was formally diagnosed with TTM and social phobia, and also has a history of bulimia. Self-ratings for mood disorders and general anxiety were not clinically significant. However, social anxiety symptoms were in the clinically significant range. His sister, who was not available for genetic testing, had also been diagnosed with TTM. She reported a history of anxiety and depressive disorders.

Of the 44 TTM nuclear families examined, two had SLITRK1 sequence variants, accounting for a frequency of 4.5%. Both changes presented with an extremely low allele frequency and occurred in a conserved region. The mutations also co-segregated with psychiatric conditions in their respective nuclear pedigrees (FIG. 4). In family DUK14044, the mother carried the mutation but had no apparent features of TTM. However, individuals affected with TTM tend to disguise the condition and a diagnosis might have been missed. Alternatively, the phenotypic penetrance is incomplete or SLITRK1 mutations may underlie an even wider spectrum of phenotypes.

Our results thus demonstrate that rare variations in the SLITRK1 gene result in disorders of the OCD spectrum.

Except as otherwise indicated, standard methods known to those skilled in the art may be used for the construction and use of nucleic acid molecules, vectors, selectable markers, cells, transgenic organisms, and the like. Such techniques are well known to those skilled in the art. See, e.g., J. Sambrook & D. Russell, Molecular Cloning: A Laboratory Manual 3rd Ed. (2001) (Cold Spring Harbor Laboratory Press; Woodbury, N.Y.) and Current Protocols In Molecular Biology, edited by F. M. Ausubel et al. (John Wiley & Sons, Inc.; Hoboken, N.J.).

All publications, patents, and patent publications cited herein are incorporated by reference in their entireties for the teachings relevant to the sentence and/or paragraph in which the reference is presented. The foregoing is illustrative of the present invention, and is not to be construed as limiting thereof. The invention is defined by the following claims, with equivalents of the claims to be included therein.

We claim:

1. A method of identifying a subject having an increased risk of developing a disorder in the OCD spectrum, the method comprising detecting in the subject a mutation in the SLITRK1 gene resulting in a mutation in the SLITRK1 amino acid sequence between the second leucine rich repeat (LRR) domain and the transmembrane domain of the SLITRK1 protein.

2. A method of diagnosing a disorder in the OCD spectrum in a subject, the method comprising detecting in the subject a mutation in the SLITRK1 gene resulting in a mutation in the SLITRK1 amino acid sequence between the second leucine rich repeat (LRR) domain and the transmembrane domain of the SLITRK1 protein.

3. The method of claim 2, wherein the mutation in the SLITRK1 gene is a missense mutation.

4. The method of claim 3, wherein the mutation comprises a missense mutation resulting in a change at position 584 and/or position 593 of the SLITRK1 amino acid sequence of SEQ ID NO:1 or a corresponding position in other SLITRK1 proteins.

5. The method of claim 4, wherein the mutation in the SLITRK1 amino acid sequence comprises a R→K mutation at position 584 and/or an S→G mutation at position 593 of the SLITRK1 amino acid sequence provided in SEQ ID NO:1 or a corresponding position in other SLITRK1 proteins.

6. The method of claim 5, wherein the mutation in the SLITRK1 amino acid sequence is the result of a G→A mutation at nucleotide 2637 and/or an A→G mutation at nucleotide 2663 of the SLITRK1 nucleotide sequence provided in SEQ ID NO:2 or a corresponding position in other SLITRK1 genes.

7. The method of claim 2, wherein the disorder is selected from the group consisting of bulimia, anorexia nervosa, anxiety, depression, skin picking, motor tics, trichotillomania, and any combination thereof.

8. The method of claim 7, wherein the disorder is trichotillomania.

9. The method of claim 2, wherein the subject is a human subject.

10. The method of claim 2, wherein the mutation is detected at the nucleic acid level.

11. The method of claim 2, wherein the mutation is detected at the amino acid level.

12. A method of identifying a subject having an increased risk of developing a disorder in the OCD spectrum, the method comprising:
(a) correlating the presence of one or more mutations in the SLITRK1 gene with an increased risk of developing the disorder, wherein the mutation in the SLITRK1 gene results in a mutation in the SLITRK1 amino acid sequence between the second leucine rich repeat (LRR) domain and the transmembrane domain of the SLITRK1 protein; and
(b) detecting the one or more mutations of step (a) in the subject, thereby identifying the subject as having an increased risk of developing the disorder.

13. A method of diagnosing a disorder in the OCD spectrum in a subject, the method comprising:
(a) correlating the presence of one or more mutations in the SLITRK1 gene with an increased risk of developing the disorder, wherein the mutation in the SLITRK1 gene results in a mutation in the SLITRK1 amino acid sequence between the second leucine rich repeat (LRR) domain and the transmembrane domain of the SLITRK1 protein; and
(b) detecting the one or more mutations of step (a) in the subject, thereby diagnosing the subject as having the disorder.

14. The method of claim 13, wherein the disorder is trichotillomania.

15. The method of claim 13, wherein the mutation is a missense mutation.

16. The method of claim 15, wherein the mutation comprises a missense mutation resulting in a change at position 584 and/or position 593 of the SLITRK1 amino acid sequence of SEQ ID NO:1 or a corresponding position in other SLITRK1 proteins.

17. The method of claim 16, wherein the mutation in the SLITRK1 amino acid sequence comprises a R→K mutation at position 584 and/or an S→G mutation at position 593 of the SLITRK1 amino acid sequence provided in SEQ ID NO:1 or a corresponding position in other SLITRK1 proteins.

18. The method of claim 17, wherein the mutation in the SLITRK1 amino acid sequence is the result of a G→A mutation at nucleotide 2637 and/or an A→G mutation at nucleotide 2663 of the SLITRK1 nucleotide sequence provided in SEQ ID NO:2 or a corresponding position in other SLITRK1 genes.

19. A method of correlating a mutation in the SLITRK1 gene with an increased risk of developing a disorder in the OCD spectrum, the method comprising:
(a) detecting in a subject with the disorder the presence of one or more mutations in the SLITRK1 gene, wherein the mutation in the SLITRK1 gene results in a mutation in the SLITRK1 amino acid sequence between the second leucine rich repeat (LRR) domain and the transmembrane domain of the SLITRK1 protein; and
(b) correlating the presence of the one or more mutations in the SLITRK1 gene of step (a) with the disorder in the subject.

20. A method of correlating a mutation in the SLITRK1 gene with a diagnosis of a disorder in the OCD spectrum, the method comprising:
(a) detecting in a subject diagnosed with the disorder the presence of one or more mutations in the SLITRK1 gene, wherein the mutation in the SLITRK1 gene results in a mutation in the SLITRK1 amino acid sequence between the second leucine rich repeat (LRR) domain and the transmembrane domain of the SLITRK1 protein; and
(b) correlating the presence of the one or more mutations in the SLITRK1 gene of step (a) with the disorder in the subject.

21. The method of claim 20, wherein the disorder is trichotillomania.

22. The method of claim 20, wherein the mutation is a missense mutation.

23. A method of correlating a mutation in the SLITRK1 gene with a good prognosis for a disorder in the OCD spectrum, the method comprising:
(a) detecting in a subject with the disorder and having a good prognosis, the presence of one or more mutations in the SLITRK1 gene; and
(b) correlating the presence of the one or more mutations in the SLITRK1 gene of step (a) with a good prognosis for the disorder.

24. A method of identifying a subject with a disorder in the OCD spectrum as having a good prognosis, the method comprising:
(a) correlating the presence of the one or more mutations in the SLITRK1 gene with a good prognosis for the disorder; and
(b) detecting the one or more mutations of step (a) in the subject, thereby identifying the subject as having a good prognosis.

25. A method of correlating a mutation in the SLITRK1 gene with a poor prognosis for a disorder in the OCD spectrum, the method comprising:
(a) detecting in a subject with the disorder and having a poor prognosis, the presence of one or more mutations in the SLITRK1 gene; and
(b) correlating the presence of the one or more mutations in the SLITRK1 gene of step (a) with a poor prognosis for the disorder.

26. A method of identifying a subject with a disorder in the OCD spectrum as having a poor prognosis, the method comprising:
(a) correlating the presence of the one or more mutations in the SLITRK1 gene with a poor prognosis for the disorder; and
(b) detecting the one or more mutations of step (a) in the subject, thereby identifying the subject as having a poor prognosis.

27. A method of correlating a mutation in the SLITRK1 gene with an effective treatment regimen for a disorder in the OCD spectrum, the method comprising:
(a) detecting in a subject with the disorder and for whom an effective treatment regimen has been identified, the presence of one or more mutations in the SLITRK1 gene; and
(b) correlating the presence of the one or more mutations of step (a) with an effective treatment regimen for the disorder.

28. A method of identifying a treatment regimen for a subject with a disorder in the OCD spectrum, the method comprising:
(a) correlating the presence of one or more mutations in the SLITRK1 gene in a test subject with the disorder for whom an effective treatment regimen has been identified; and
(b) detecting the one or more mutations of step (a) in the subject, thereby identifying a treatment regimen for the subject.

29. The method of claim 28, wherein the method comprises detecting in the subject a mutation in the SLITRK1 gene resulting in a mutation in the SLITRK1 amino acid sequence between the second leucine rich repeat (LRR) domain and the transmembrane domain of the SLITRK1 protein.

30. The method of claim 29, wherein the mutation is a missense mutation.

31. The method of claim 30, wherein the mutation comprises a missense mutation resulting in an change at position 584 and/or position 593 of the SLITRK1 amino acid sequence of SEQ ID NO:1 or a corresponding position in other SLITRK1 proteins.

32. The method of claim 31, wherein the mutation in the SLITRK1 amino acid sequence comprises a R→K mutation at position 584 and/or an S→G mutation at position 593 of the SLITRK1 amino acid sequence provided in SEQ ID NO:1 or a corresponding position in other SLITRK1 proteins.

33. The method of claim 31, wherein the mutation in the SLITRK1 amino acid sequence is the result of a G→A mutation at nucleotide 2637 and/or an A→G mutation at nucleotide 2663 of the SLITRK1 nucleotide sequence provided in SEQ ID NO:2 or a corresponding position in other SLITRK1 genes.

34. A method of identifying a candidate compound for the treatment of a disorder in the OCD spectrum, the method comprising:
(a) contacting a SLITRK1 protein or functional portion thereof with a test compound; and
(b) detecting binding of the compound to the SLITRK1 protein and/or modulation of the SLITRK1 protein activity,
wherein a compound that binds to the SLITRK1 protein and/or modulates SLITRK1 protein activity as compared with the activity in the absence of the test compound is identified as a candidate compound for the treatment of the disorder.

35. The method of claim 34, wherein the SLITRK1 protein comprises a mutation associated with the disorder.

36. The method of claim 34, wherein the SLITRK1 protein does not comprise a mutation associated with the disorder, and optionally is a wild-type SLITRK1 protein.

37. A method of identifying a candidate compound for the treatment of a disorder in the OCD spectrum, the method comprising:
(a) contacting a SLITRK1 nucleic acid or functional portion thereof with a test compound; and
(b) detecting binding of the compound to the SLITRK1 nucleic acid and/or modulation of the SLITRK1 nucleic acid activity,
wherein a compound that binds to the SLITRK1 nucleic acid and/or modulates SLITRK1 nucleic acid activity as compared with the activity in the absence of the test compound is identified as a candidate compound for the treatment of the disorder.

38. The method of claim 37, wherein the SLITRK1 nucleic acid comprises a mutation associated with the disorder.

39. The method of claim 37, wherein the SLITRK1 nucleic acid does not comprise a mutation associated with the disorder, and optionally is a wild-type SLITRK1 nucleic acid.


SEQUENCE LISTING (11 sequences)

SEQ ID NO: 1 - protein, 696 aa, Homo sapiens
MLLWILLLETSLCFAAGNVTGDVCKEKICSCNEIEGDLHVDCEKKGFTSLQRFTAPTSQFYHLFLHGNSLTRLFPNEFANFYNAVSLHMENNGLHEIVPGAFLGLQLVKRLHINNNKIKSFRKQTFLGLDDLEYLQADFNLLRDIDPGAFQDLNKLEVLILNDNLISTLPANVFQYVPITHLDLRGNRLKTLPYEEVLEQIPGIAEILLEDNPWDCTCDLLSLKEWLENIPKNALIGRVVCEAPTRLQGKDLNETTEQDLCPLKNRVDSSLPAPPAQEETFAPGPLPTPFKTNGQEDHATPGSAPNGGTKIPGNWQIKIRPTAAIATGSSRNKPLANSLPCPGGCSCDHIPGSGLKMNCNNRNVSSLADLKPKLSNVQELFLRDNKIHSIRKSHFVDYKNLILLDLGNNNIATVENNTFKNLLDLRWLYMDSNYLDTLSREKFAGLQNLEYLNVEYNAIQLILPGTFNAMPKLRILILNNNLLRSLPVDVFAGVSLSKLSLHNNYFMYLPVAGVLDQLTSIIQIDLHGNPWECSCTIVPFKQWAERLGSEVLMSDLKCETPVNFFRKDFMLLSNDEICPQLYARISPTLTSHSKNSTGLAETGTHSNSYLDTSRVSISVLVPGLLLVFVTSAFTVVGMLVFILRNRKRSKRRDANSSASEINSLQTVCDSSYWHNGPYNADGAHRVYDCGSHSLSD

SEQ ID NO: 2 - DNA, 5201 bp, Homo sapiens
cgcgctctcttcctccctcagacaactcgccccccgccctccgcccccctccacgtaattccgaaagagcagaagaaagagaaggagaacaggaaaagaagagctagtaagcgagagcgagagcacagaaaagaaaaaaaaaagccttaagaggaccgaaggggaggaaaggaaaaggatggacaaccacaaaacgcagcgattgcggaaattttccagcgccattggctgggcagcgtgagtccttcggtcgggcgtgatttcagcaccgggggaactggacagcacctcggggggacttctgggcaacccgcaaccacagcaagaactccaccagcagcctcaacaacagaagccgcggaaaaccctgctttgtatcagagaggcaaggtcagtccgacgcacagccatgcacaggcagtgcgcctgtactacgctgcaaaccctctgcttgtttctctaacatgcacttgcttctaattactagcattgtttcatttctgatcagtgaagatcagtagatgagattctgtaagggtgtacttttaatttatatgtatatatttaacttctttttctgttatttttaaagtgttgtgggggagtggggtttttttcctacttttttttttttttttttttctttgcttgccttgcactacgtgcctggatagtttgtggatataattattgactggcgtctgggctattgcagtgcgggggggttagggaggaaggaatccacccccacccccccaaacccttttcttctcctttcctggcttcggacattggagcactaaatgaacttgaattgtgtctgtggcgagcaggatggtcgctgttactttgtgatgagatcggggatgaattgctcgctttaaaaatgctgctttggattctgttgctggagacgtctctttgttttgccgctggaaacgttacaggggacgtttgcaaagagaagatctgttcctgcaatgagatagaaggggacctacacgtagactgtgaaaaaaagggcttcacaagtctgcagcgtttcactgccccgacttcccagttttaccatttatttctgcatggcaattccctcactcgacttttccctaatgagttcgctaacttttataatgcggttagtttgcacatggaaaacaatggcttgcatgaaatcgttccgggggcttttctggggctgcagctggtgaaaaggctgcacatcaacaacaacaagatcaagtcttttcgaaagcagacttttctggggctggacgatctggaatatctccaggctgattttaatttattacgagatatagacccgggggccttccaggacttgaacaagctggaggtgctcattttaaatgacaatctcatcagcaccctacctgccaacgtgttccagtatgtgcccatcacccacctcgacctccggggtaacaggctgaaaacgctgccctatgaggaggtcttggagcaaatccctggtattgcggagatcctgctagaggataacccttgggactgcacctgtgatctgctctccctgaaagaatggctggaaaacattcccaagaatgccctgatcggccgagtggtctgcgaagcccccaccagactgcagggtaaagacctcaatgaaaccaccgaacaggacttgtgtcctttgaaaaaccgagtggattctagtctcccggcgccccctgcccaagaagagacctttgctcctggacccctgccaactcctttcaagacaaatgggcaagaggatcatgccacaccagggtctgctccaaacggaggtacaaagatcccaggcaactggcagatcaaaatcagacccacagcagcgatagcgacgggtagctccaggaacaaacccttagctaacagtttaccctgccctgggggctgcagctgcgaccacatcccagggtcgggtttaaagatgaactgcaacaacaggaacgtgagcagcttggctgatttgaagcccaagctctctaacgtgcaggagcttttcctacgagataacaagatccacagcatccgaaaatcgcactttgtggattacaagaacctcattctgttggatctgggcaacaataacatcgctactgtagagaacaacactttcaagaaccttttggacctcaggtggctatacatggatagcaattacctggacacgctgtcccgggagaaattcgcggggctgcaaaacctagagtacctgaacgtggagtacaacgctatccagctcatcctcccgggcactttcaatgccatgcccaaactgaggatcctcattctcaacaacaacctgctgaggtccctgcctgtggacgtgttcgctggggtctcgctctctaaactcagcctgcacaacaattacttcatgtacctcccggtggcaggggtgctggaccagttaacctccatcatccagatagacctccacggaaacccctgggagtgctcctgcacaattgtgcctttcaagcagtgggcagaacgcttgggttccgaagtgctgatgagcgacctcaagtgtgagacgccggtgaacttctttagaaaggatttcatgctcctctccaatgacgagatctgccctcagctgtacgctaggatctcgcccacgttaacttcgcacagtaaaaacagcactgggttggcggagaccgggacgcactccaactcctacctagacaccagcagggtgtccatctcggtgttggtcccgggactgctgctggtgtttgtcacctccgccttcaccgtggtgggcatgctcgtgtttatcctgaggaaccgaaagcggtccaagagacgagatgccaactcctccgcgtccgagattaattccctacagacagtctgtgactcttcctactggcacaatgggccttacaacgcagatggggcccacagagtgtatgactgtggctctcactcgctctcagactaagaccccaaccccaataggggagggcagagggaaggcgatacatccttccccaccgcaggcaccccgggggctggaggggcgtgtacccaaatccccgcgccatcagcctggatgggcataagtagataaataactgtgagctcgcacaaccgaaagggcctgaccccttacttagctccctccttgaaacaaagagcagactgtggagagctgggagagcgcagccagctcgctctttgctgagagccccttttgacagaaagcccagcacgaccctgctggaagaactgacagtgccctcgccctcggccccggggcctgtggggttggatgccgcggttctatacatatatacatatatccacatctatatagagagatagatatctatttttcccctgtggattagccccgtgatggctccctgttggctacgcagggatgggcagttgcacgaaggcatgaatgtattgtaaataagtaactttgacttctgacaaaaaacaaaaagtgctgcatggctcgcatggaatccacgcgctccagggactctgcccgcccccgcgactggagacggcatctcgttcacagcacccaccctcttacctgataagttccatcgtatcaaactttctataaacaaaatacagtataatcagaaagtgccatttcgccattatttgtgatcggtaggcagttcagagcataagttaactgtgaaaaaaatgtaaaggttttatttaggacatttgcatggctagtcatcagtccattttatgagttaacaatgtattttgttgagggaagtttttaggggttgttttgggttcttttattttgatggtgatgttttattttattttatttttttcagggggtcttttttttaatacatatccaataatgccttccatctgaatgtaaaataagtacccatgatttctattatagtatcagtgtaattatttaaaaaatgattttgaggcagttaagcatgaccaattaatgtcactctagtgcttaggctgcgatcctatggtagcaattctgtgctggtataaatcttacttataaagtaggaaaagagaaccgaggaagcacgtgaaacttactaattctattcgaggattttataatggcatattttttcagtattaaagcgaaaatgttttcaactctgggtccttacctttttccagcttcatatttgcaagtggtaaattggatttgcggtggaagagacaggggagggaaacggttggggttagatcccttcctgagctacattaaggctctttctctaatcgccttacttagctttttaccctttaagtagctcctcttccctcgcccccaccctctaccccacccccaccttcgctcagactttaccggctttccccagtccataaaggtcttgccccaacactcaccccttctttttttcccctctccaaatgcagcagtgaatccctttattaatactggaaatccctctctgctgcttttgttggtgctgcccacactgcagatatattaaggatgttaggagagatttgatttaattgactctgcctagataggtctcattaaacagagtggagatttcattggtcagcactcctcaatgaaagacagacctaatgactggcatttgagatgctgctggcattttgaattcaacatctgctgaaaacggtaaaactaattagtgcccacccaccctccccgccccagcaactgcatattgaaatttgttaaagcactcatctttatggaaattaatcattatcctaaagaagtgtttctctcccatcatccggatttctggttgtggcccagcaattaacaaaaacagcttcaactgttcgaattttatgaaccaatgtaactctggcctcaatcatattcctctgggatttctaaacagcagttaagctacaaaaagcaaacaaaaccacacatattgatggagtctgcattccaccacatatccacccttgagaagtatgtcaaaagactgcagactatagatttttttttaatataggattataaatcagctagtgaaagacctcagagcagttgtaagtagatctgccatctagaactcatattctaaagggaagtgatttctcagaacagtgatgttctggaatatgtattatttattttaacacttttttaataaaatctttattataaaccaaaaaaaaaaaaaaaa

SEQ ID NO: 3 - DNA, 13 bp, Homo sapiens
acgctaggatctc

SEQ ID NO: 4 - DNA, 13 bp, Homo sapiens
  misc_feature (7)..(7): n represents a
acgctangatctc

SEQ ID NO: 5 - DNA, 13 bp, Homo sapiens
tcgcacagtaaaa

SEQ ID NO: 6 - DNA, 13 bp, Homo sapiens
  misc_feature (7)..(7): n represents g
tcgcacngtaaaa

SEQ ID NO: 7 - protein, 37 aa, Homo sapiens
PVNFFRKDFMLLSNDEICPQLYARISPTLTSHSKNST

SEQ ID NO: 8 - protein, 37 aa, Mus musculus
PVNFFRKDFMLLSNEEICPQLYARISPTLTSHSKNST

SEQ ID NO: 9 - protein, 37 aa, Bos taurus; identical to SEQ ID NO: 7

SEQ ID NO: 10 - protein, 37 aa, Canis familiaris; identical to SEQ ID NO: 7

SEQ ID NO: 11 - protein, 35 aa, Tetraodon nigroviridis
PEEFWKRDFRYVRNDRMCPKLYDKVPPTALSKNST